(19) United States
(12) Patent Application Publication
(10) Pub. No.: US 2010/0022813 A1
(43) Pub. Date: Jan. 28, 2010

(54) Process for Reducing Ethylbenzene Content from an Aromatic Stream

(75) Inventors: James R. Butler, League City, TX (US); Joseph E. Pelati, Houston, TX (US); Darek Wachowicz, Friendswood, TX (US); Taylor Rives, Houston, TX (US); Xin Xiao, Augusta, GA (US)

Correspondence Address: FINA TECHNOLOGY INC, PO BOX 674412, HOUSTON, TX, 77267-4412, US

(73) Assignee: Fina Technology, Inc., Houston, TX (US)

(21) Appl. No.: 12/181,276

(22) Filed: Jul. 28, 2008

(51) Int. Cl. C07C 1/00; C07C 5/52

(52) U.S. Cl. 585/469; 585/470; 585/475

(57) ABSTRACT

A method of reducing the ethylbenzene content in a stream containing xylene is disclosed. The method includes the reaction of ethylbenzene, such as a disproportionation or transalkylation reaction, to produce benzene and other hydrocarbon compound and can include the separation of at least a portion of the resulting benzene and other hydrocarbon compounds to produce a xylene stream having reduced ethylbenzene content.

### CROSS-REFERENCE TO RELATED APPLICATIONS

Not applicable.

### FIELD

This invention relates to aromatic compounds and the production of commercial grade product streams of aromatic compounds.

### BACKGROUND

Pyrolysis gasoline (also referred to as “pygas”) is a liquid byproduct of the steam cracking process of hydrocarbons. Crude oil fractions such as straight run naphtha from a crude oil still are conventionally steam cracked in an olefins unit to produce light olefins and aromatics. Pygas is a highly unsaturated hydrocarbon mixture (carbon range of about C₅ to C₁₄) that is generally rich in dienes, olefins, and aromatics.

Pygas can be further processed to produce other products by using one or more of hydrotreating, solvent extraction, distillation and other processes known in the art. A mixed xylene stream is one product that can be obtained from pygas, but may contain ethylbenzene in significant quantities. A mixed xylene stream can include any of m-xylene, o-xylene and p-xylene, or combinations thereof.

Xylene has a number of uses in the chemical industry. High purity xylene product can be produced in processes well known in the industry, such as typical BTX (Benzene Toluene Xylene) units. Other processes by which xylene can be generated, such as the thermal cracking of naphtha, may also produce byproducts such as ethylbenzene. A product stream comprising xylene and ethylbenzene may be used in various ways, such as for fuel blending, but may have a higher value as a commercial xylene stream if the composition is within certain product specifications. For a commercial grade xylene product the ethylbenzene content should be less than about 18%, which can require ethylbenzene be removed from the xylene stream if its content is above this threshold. It can be difficult to physically separate ethylbenzene from xylene by typical methods such as distillation because they have such similar boiling points and molecular weights; xylene having a boiling point of about 139° C. and ethylbenzene having a boiling point of about 136° C.

In view of the above, it would be desirable to have an effective method to reduce the ethylbenzene content in a product stream containing xylene and ethylbenzene.

### SUMMARY

Embodiments of the present invention include a method of reducing the ethylbenzene content in a stream containing xylene by providing a reaction zone containing a catalyst and introducing a feed stream comprising xylene and ethylbenzene to the reaction zone. At least a portion of the ethylbenzene converts to produce benzene and/or other hydrocarbon compounds other than ethylbenzene.

A first product stream can be removed from the reaction zone, the first product stream having reduced ethylbenzene content than the feed stream. At least a portion of the benzene and other hydrocarbon compounds other than ethylbenzene are removed from the first product stream to make a second product stream that now has lower ethylbenzene content than the feed stream.

The xylene can comprise at least 25% by total weight of the feed stream. The ethylbenzene can comprise at least 25% by total weight of the feed stream or can comprise at least 40% by total weight of the feed stream. The ethylbenzene can comprise less than 25% by total weight of the second product stream or can comprise less than 18% by total weight of the second product stream. The xylene can comprise more than 75% by total weight of the second product stream. The catalyst can have an average pore size of 6.0 angstroms or greater. The second product stream can be within the composition specifications of a commercial grade mixed xylene product.

The method can further include blending the second product stream with a third product stream containing xylene to make a fourth product stream, wherein the fourth product stream has a lower ethylbenzene content than the second product stream. The fourth product stream can have an ethylbenzene content of less than 18 wt %. The fourth product stream can be within the composition specifications of a commercial grade mixed xylene product.

The catalyst can be disproportionation catalyst. The disproportionation catalyst can be a zeolite catalyst, can be a zeolite mordenite catalyst, can be a metal modified zeolite mordenite catalyst, or can be a zeolite nickel-mordenite catalyst. The mordenite catalyst can be a nickel-containing mordenite catalyst containing from 0.5% to 1.5% by weight nickel. The reaction zone can be operated at a temperature of from 65° C. to 500° C. and a pressure of between 200 psig to 1,000 psig.

The catalyst can be transalkylation catalyst. The transalkylation catalyst can be a zeolite catalyst, for example can be a zeolite Y catalyst, or a zeolite beta catalyst, or combinations thereof. The reaction zone can be operated at a temperature of from 180° C. to 280° C. and a pressure of between 400 psig to 800 psig.

An alternate embodiment of the present invention is a method of processing pyrolysis gasoline to produce a commercial grade xylene product. The method includes providing a pyrolysis gasoline stream and separating a first product stream containing mixed xylene and ethylbenzene from the pyrolysis gasoline stream. The first product stream is introduced to a reaction zone containing a disproportionation catalyst at disproportionation reaction conditions. At least a portion of the ethylbenzene of the first product stream is reacted to produce lighter compounds such as benzene and ethylene and/or heavier compounds such as ethylxylene. A second product stream having reduced ethylbenzene content than the first product stream is removed from the reaction zone. At least a portion of the lighter compounds such as benzene and ethylene and/or heavier compounds such as ethylxylene are removed from the second product stream to make a third product stream having a reduced ethylbenzene content than the first product stream.

The third product stream can have an ethylbenzene content of less than 25% by total weight. The method can also include blending the third product stream with a fourth product stream containing xylene to make a fifth product stream, the fifth product stream having a lower percentage of ethylbenzene than the third product stream. The fifth product stream can have an ethylbenzene content of less than 18% by total weight.

An alternate embodiment of the present invention is a method of converting a feed of heavy aromatics composed primarily of xylene and ethylbenzene, which involves providing a reaction zone containing a nickel-mordenite catalyst and introducing a first feed of substantially pure toluene feedstock into the reaction zone so that the first feed contacts the catalyst under initial reaction zone conditions selected for the disproportionation of substantially pure toluene to obtain a target toluene conversion between 30% and 55%. A second feed comprising xylene and ethylbenzene is introduced while the reaction zone is at the reaction zone conditions selected for the disproportionation of the pure toluene. The reactor conditions are then adjusted to control the conversion product composition. Conversion products are removed from the reaction zone wherein the ethylbenzene content in the conversion products is reduced as compared to the second feed.

The mordenite catalyst can be a nickel-containing mordenite catalyst containing from 0.5% to 1.5% by weight nickel. The reaction zone can be operated at a temperature of from 250° C. to 500° C. and at a pressure of at least 200 psig.

The conversion products can be separated to obtain a first product stream composed primarily of xylene and ethylbenzene wherein the first product stream has an ethylbenzene content of less than 25% by total weight. The first product stream can be blended with a second product stream containing xylene to make a third product stream, wherein the third product stream has a lower percentage of ethylbenzene than the first product stream. The third product stream can have an ethylbenzene content of less than 18% by total weight. The third product stream can have a composition within the specifications of a commercial grade xylene stream.

An alternate embodiment can be a method of processing pyrolysis gasoline to produce a commercial grade xylene product. The method includes providing a pyrolysis gasoline stream and separating a first product stream containing mixed xylene and ethylbenzene from the pyrolysis gasoline stream. The first product stream is introduced to a reaction zone containing a transalkylation catalyst at transalkylation reaction conditions. At least a portion of the ethylbenzene of the first product stream is reacted to produce benzene and diethylbenzene. A second product stream having reduced ethylbenzene content than the first product stream is removed from the reaction zone. At least a portion of the benzene and diethylbenzene is removed from the second product stream to make a third product stream having a reduced ethylbenzene content than the first product stream.

The third product stream can have an ethylbenzene content of less than 25% by total weight. The method can also include blending the third product stream with a fourth product stream containing xylene to make a fifth product stream, the fifth product stream having a lower percentage of ethylbenzene than the third product stream. The fifth product stream can have an ethylbenzene content of less than 18% by total weight.

### BRIEF DESCRIPTION OF DRAWINGS

FIG. 1 illustrates experimental results obtained from one study regarding the embodiment of the present invention utilizing a disproportionation reaction.

FIG. 2 provides a summary of the experimental reaction conditions and the resulting product composition of the study with results shown in FIG. 1.

FIG. 3 illustrates experimental results obtained from one study regarding the embodiment of the present invention utilizing a transalkylation reaction.

FIG. 4 provides a summary of the experimental reaction conditions and the resulting product composition of the study with results shown in FIG. 3.

FIG. 5 illustrates experimental results obtained from one study regarding the embodiment of the present invention utilizing a transalkylation reaction.

FIG. 6 illustrates an embodiment of a separation process that can be used with the present invention.

### DETAILED DESCRIPTION

For a xylene product stream to be considered a commercial grade xylene product, the ethylbenzene content should be less than 18%. The separation of ethylbenzene from xylene can be difficult due to the similarity of physical properties the two compounds have. The boiling point of xylene is about 139° C., while the boiling point of ethylbenzene is about 136° C. With a boiling point differential of only about 3° C., distillation separation is generally not practical. To facilitate the physical separation of ethylbenzene from xylene, the physical properties of the ethylbenzene can be altered by a chemical conversion of the ethylbenzene to other compounds such as benzene, diethylbenzene, ethylxylene or toluene. The boiling point of benzene is about 80° C., the boiling point of diethylbenzene is about 184° C., while the boiling point for toluene is about 111° C. The boiling point differential with xylene is only 3° C. for ethylbenzene while it is 59° C. for benzene, 45° C. for diethylbenzene, and 28° C. for toluene. Once ethylbenzene molecules are converted to benzene or other light components such as ethylene, or heavier components such as ethylxylene, they can be physically separated from the xylene by normal separation methods such as boiling point distillation. This can result in a reduction of the amount of ethylbenzene present in the processed stream and enable the remaining xylene stream with reduced ethylbenzene content to be sold as a commercial grade xylene product, or if the ethylbenzene content is still above the specification, facilitate its blending with an existing xylene stream having a lower ethylbenzene content, such as a xylene stream from a BTX unit.

A variety of disproportionation reactions are employed in petroleum refining operations to interchange the substituents on aromatic hydrocarbon rings. One such reaction commonly employed is the Toluene Disproportionation (TDP) reaction. The TDP reaction, which typically takes place in the presence of molecular hydrogen, is a well-known reaction in which two equivalents of toluene are converted into benzene and xylene.

Disproportionation reactions utilizing various catalysts have been employed using a variety of feed streams. For instance, U.S. Pat. No. 5,475,180 to Shamshoum, incorporated by reference herein in its entirety, demonstrates a TDP reaction using a nickel-promoted mordenite catalyst can be employed where pure toluene is mixed with a heavy aromatic containing feedstream. U.S. Pat. No. 6,504,076 to Xiao, incorporated by reference herein in its entirety, demonstrates that a disproportionation reaction using a nickel, palladium, or platinum modified mordenite catalyst can be employed with a feed of heavy aromatics containing primarily C₈+ alkylaromatic compounds to produce benzene, toluene, and xylene.

Although the above references disclose the disproportionation reaction may be employed in the presence of heavy aromatic containing feed streams, none disclose a method wherein the disproportionation reaction is employed to reduce the content of ethylbenzene in a mixed xylene stream. Such a reaction would be of value to the industry because it would provide an effective means to reduce the ethylbenzene content of such a mixed stream.

Mordenite is a molecular sieve catalyst that is useful in reactions of alkylaromatic compounds such as in a TDP reaction. Mordenite is a crystalline aluminosilicate zeolite exhibiting a network of silicon and aluminum ions interlinked by oxygen atoms within the crystalline structure. Mordenite can be found naturally occurring or synthetically created. A suitable mordenite catalyst may have a silica-to-alumina ratio of between 5:1 and 50:1.

It is a common practice to supplement aluminum deficient Mordenite catalysts with a catalytically active metal component. Group VIIB and Group VIII metals such as molybdenum, tungsten, chromium, iron, nickel, cobalt, platinum, palladium, ruthenium, rhodium, osmium, and iridium have all been used as supplements. The inclusion of the metal group can increase activity and catalyst life. Metal modifications to the mordenite catalyst may include nickel, palladium, and platinum.

Nickel is a metallic ion suitable for use in modification of the mordenite catalyst. It is known that low nickel content mordenite catalysts provide toluene conversion and selectivity to xylenes and benzene. The nickel content of the mordenite catalyst is expressed in terms of the amount of nickel based upon the amount of zeolite present without reference to a binder, which will normally be employed to form the particulate catalyst actually incorporated into the reaction zone. In one non-limiting example suitable nickel content for the present invention can range from 0.5 wt % to 1.5 wt %.

An aspect of the present invention involves the disproportionation (and/or dealkylation), desirably in a vapor phase, of ethylbenzene in a xylene product stream to produce other hydrocarbon compounds such as for example, benzene, ethylene and ethylxylene. The feedstock supplied to the reactor can comprise a mix of xylene and ethylbenzene, such as for example a xylene stream from a naphtha cracker and/or from pyrolysis gasoline processing. The reaction may occur over a variety of temperature and pressure conditions. The reaction can be carried out under conditions permitting the ethylbenzene and xylene to be in a vapor phase. Specifically the temperature may range from 65° C. to 600° C. and pressures of 1,000 psig or less. In one embodiment the temperature may range from 200° C. to 500° C. and a pressure of 250 psig to 800 psig. In one embodiment the temperature may range from 350° C. to 450° C. and a pressure of 500 psig to 700 psig.

The reaction of ethylbenzene to other compounds such as benzene may occur over a variety flow rates that are system specific and are not limiting restrictions to the invention. The lower limit for flow rate will not be reaction driven but generally is an economic determination. In general the upper limit for flow rate is where the disproportionation reaction is not providing the conversion that is required. In one embodiment the LHSV rate can range from 0.1 hr⁻¹ to 1,000 hr⁻¹. In alternate embodiments the LHSV rate can range from 0.1 hr⁻¹ to 200 hr⁻¹ from 1 hr⁻¹ to 50 hr⁻¹, from 1 hr⁻¹ to 25 hr⁻¹, from 1 hr⁻¹ to 10 hr⁻¹ or from 1 hr⁻¹ to 5 hr⁻¹.

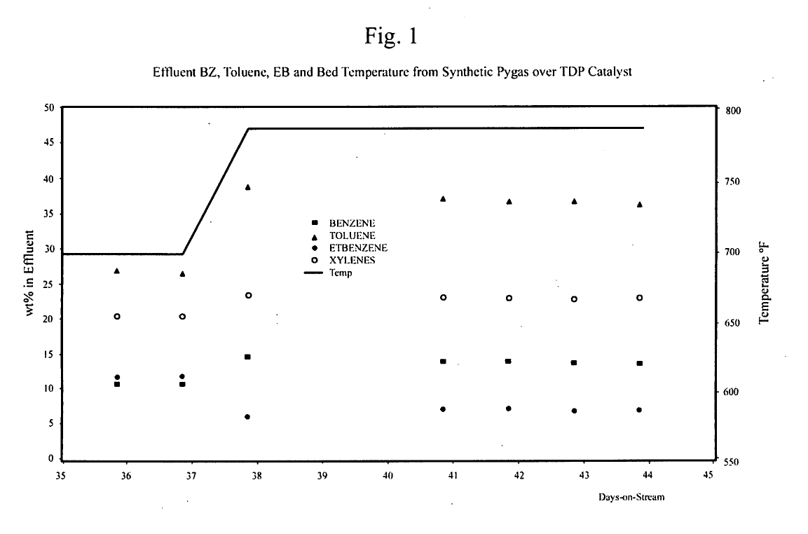

FIG. 1 illustrates experimental results from one bench reactor study. The initial ethylbenzene concentration in the mixed xylene feedstock is approximately 46%. The feedstock is fed to a reaction containing a commercially available molecular sieve nickel-mordenite zeolite catalyst from Zeolyst International known as Zeolyst CP-751. After processing at 369° C. the effluent contained a toluene concentration of approximately 27% and a benzene concentration of approximately 11%. The ethylbenzene concentration was reduced to approximately 12%, and the xylene concentration was reduced from approximately 52% to approximately 21%.

After two days the temperature was increased to 418° C. The temperature increase resulted in an increase in the production of lighter components such as benzene, toluene and non-aromatics. The temperature increase also provided a reduction in heavier components such as ethyl-toluene, tri-methyl-benzene, and di-ethyl-benzene. The effluent contained an increased toluene concentration of approximately 37% and an increased benzene concentration of approximately 14%. The ethylbenzene concentration further decreased to approximately 7% while the xylene concentration increased from approximately 21% to approximately 23%. The pressure of this reaction was at 591 psig and the LHSV rate is 3 hr⁻¹ (0.96 mL/min).

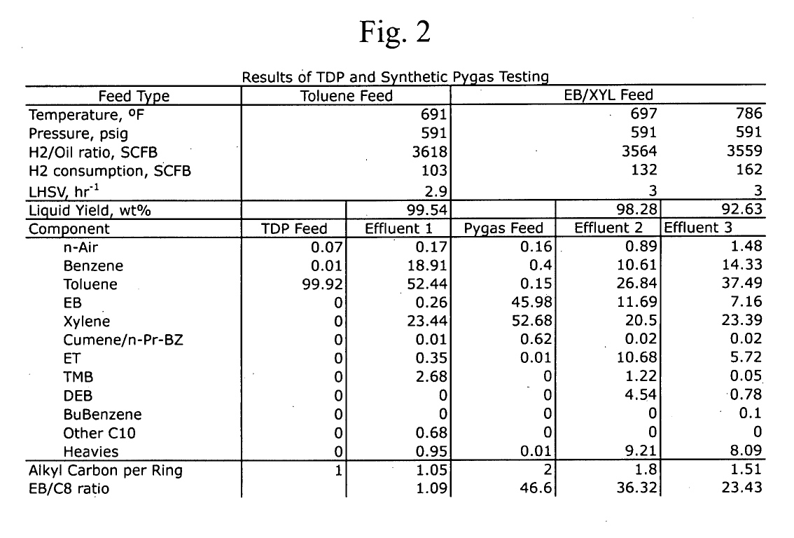

The nickel-mordenite disproportionation catalyst was in TDP service for 35 days. On day 35 the feed was changed from toluene to a feed of about 53% xylene and about 46% ethylbenzene. It can be seen in FIG. 1 that the data shown is for days 36 through 44 of on-stream flow or nine days of the heavy EB/xylene feed. The results shown in FIG. 1 indicate a stable reaction with no indication of catalyst deactivation. FIG. 2 provides a summary of the experimental reaction conditions and the resulting product composition.

The reaction may be catalyzed through the use of any suitable disproportionation catalyst, such as any suitable molecular sieve catalyst or any suitable molecular sieve zeolite catalyst. The particular disproportionation catalyst or combination thereof that is utilized is not a limitation on the scope of the invention. In an embodiment the disproportionation reaction is carried out in the gas phase and the catalyst used has a pore size sufficient to accommodate the molecular size of the reactants and products. Typically zeolite catalysts having pore sizes of 6.0 angstroms or greater are effective for gas phase disproportionation.

There should not be free water in the feed if possible, as water may have undesirable effects on certain catalysts that can be used in the present invention, although a disproportionation catalyst that is suitable for use with free water or with high water content may be used. If required, the feed may be passed through a dehydration unit to remove or reduce the water content, if any, present in the feed.

Following the disproportionation reaction, the output of the disproportionation reactor may be routed to a separation process to remove the produced benzene and toluene from the xylene stream. The separation process can take a variety of forms, for example, boiling point distillation is a commonly employed separation technique within the industry. The boiling point of a compound is the temperature at which the vapor pressure of the liquid phase of a compound equals the external pressure acting on the surface of the liquid. Compounds generally have different, well-defined boiling points. For instance xylene has a boiling point of approximately 139° C. while benzene has a boiling point of approximately 80° C. and toluene has a boiling point of approximately 111° C. This indicates that xylene will boil at a significantly higher temperature than benzene and toluene, thus providing a basis for separation of the components of the resulting stream.

An aspect of the present invention involves the transalkylation, desirably liquid phase transalkylation, of ethylbenzene in a xylene product stream to produce benzene and polyethylbenzene. The feedstock supplied to the reactor comprises a mixture of xylene and ethylbenzene, such as for example a xylene stream from a naphtha cracker and/or from pygas processing. The transalklyation reaction may occur over a variety of temperature and pressure conditions. The transalkylation reaction can be carried out under conditions permitting the ethylbenzene and xylene to remain in liquid phase. Specifically, the temperature may range from 65° C. to 290° C. and pressures of from 1,000 psig or less. In one embodiment the temperature may range from 100° C. to 290° C. and pressures of from 200 psig to 800 psig. In an alternate embodiment the temperature may range from 180° C. to 280° C. and pressures of from 400 psig to 800 psig.

The transalkylation reaction of ethylbenzene to benzene and polyethylbenzene may occur over a variety flow rates that are system specific and are not limiting restrictions to the invention. The lower limit for flow rate will not be reaction driven but generally is an economic determination. In general the upper limit for flow rate is where the transalkylation reaction is not providing the conversion that is required. In one embodiment the LHSV rate can range from 0.1 hr⁻¹ to 1,000 hr⁻¹. In alternate embodiments the LHSV rate can range from 0.1 hr⁻¹ to 200 hr⁻¹ from 1 hr⁻¹ to 50 hr⁻¹, from 1 hr⁻¹ to 25 hr⁻¹ or from 1 hr⁻¹ to 10 hr⁻¹.

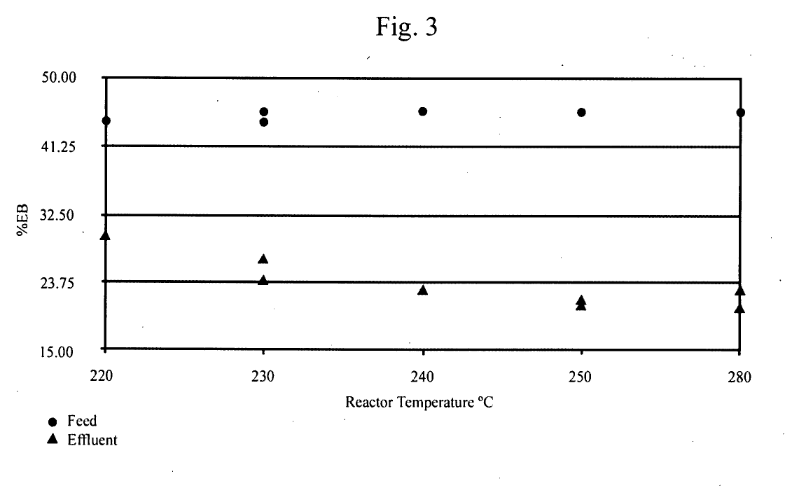

FIG. 3 illustrates experimental results from one bench reactor study. The initial ethylbenzene concentration in the mixed xylene feedstock is approximately 45%. The feedstock is fed to a reactor containing a molecular sieve zeolite catalyst. As the temperature of the reactor is increased, the ethylbenzene content of the effluent is decreased. Ultimately the ethylbenzene effluent concentration falls to approximately 20% at a temperature of 260° C. A further reduction in ethylbenzene concentration may be prohibited due to reaching equilibrium at these specific reaction conditions. The pressure of this reaction is 650 psig and the LHSV rate is 5 hr⁻¹ (1.6 mL/min).

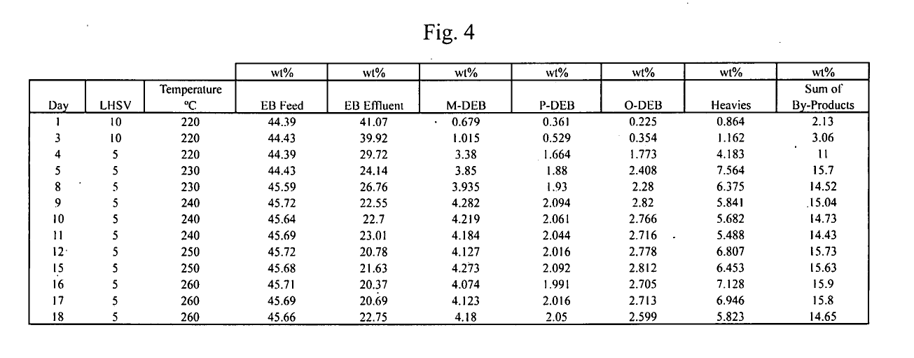

FIG. 4 provides a summary of the experimental reaction conditions and the resulting product compositions.

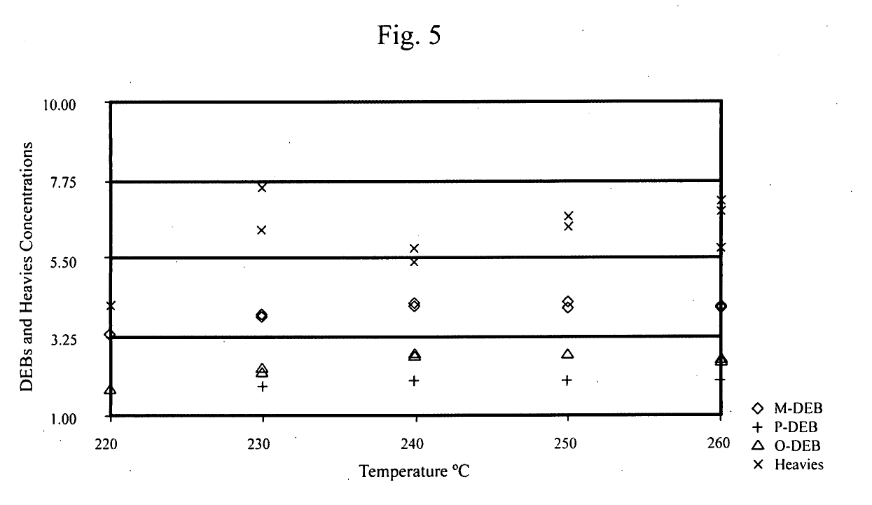

The transalkylation reaction converts the ethylbenzene in the mixed xylene stream into benzene and a variety of polyethylated aromatics such as m-diethylbenzene, o-diethylbenzene, p-diethylbenzene, and heavier aromatic compounds such as triethylbenzene, ethylxylene for example. FIG. 5 illustrates the increase in percentage of polyethylbenzene as a function of temperature. At 240° C. the m-, o-, and p-diethylbenzenes appear to reach equilibrium, although this is not the case for the heavier aromatic compounds formed during the reaction. At temperatures of 250° C. the effluent becomes yellow and further darkens at a temperature of 260° C. indicating an increase in by-product formation.

The reaction may be catalyzed through the use of any suitable transalkylation catalyst, such as any suitable molecular sieve catalyst or any suitable molecular sieve zeolite catalyst. The particular transalkylation catalyst or combination thereof that is utilized is not a limitation on the scope of the invention. In an embodiment the transalkylation reaction is carried out in the liquid phase, wherein the zeolite used should have a pore size sufficient to accommodate the liquid reactants and products. Typically zeolite catalysts having pore sizes of 6.0 angstroms or greater are effective for liquid phase transalkylation.

Zeolite beta catalysts are suitable for use in the present invention and are well known in the art. Zeolite beta catalysts typically have a silica/alumina molar ratio (expressed as SiO₂/Al₂O₃) of from 10 to 200, or 20 to 150, for example. These catalysts are characterized by having a high surface area of at least 600 m²/g based upon the crystalline form without any regard to supplemental components such as binders. The formation of zeolite beta catalysts is further described in U.S. Pat. No. 3,308,069 to Waslinger et al and U.S. Pat. No. 4,642,226 to Calvert et al, which are incorporated by reference herein.

Zeolite Y catalysts are suitable for use in the present invention and are well known in the art. A zeolite Y-84 catalyst was used to obtain the experimental results in FIGS. 1 and 2. Members of the zeolite Y family typically have a silica/alumina molar ratio between 2:1 and 80:1. In one specific embodiment the silica/alumina molar ratio is in the range of 3:1 to 15:1. In the hydrogen form, a zeolite Y catalyst will typically exhibit a pore size of between 5 and 25 angstroms, such as for example between 5 and 15 angstroms, or between 5 and 10 angstroms. The surface area is typically in excess of 500 m²/g and in one example is between the range of 700 to 1,000 m²/g. The formation of zeolite Y is further described in U.S. Pat. No. 4,185,040 to Ward et al, which is incorporated by reference herein.

Other transalkylation catalysts that may be suitable in the present invention include zeolite MCM-22, zeolite MCM-36, zeolite MCM-49, or zeolite MCM-56, for example.

There should not be free water in the feed if possible, as water may have undesirable effects on certain catalysts that can be used in the present invention, although a transalkylation catalyst that is suitable for use with free water or with high water content may be used. If required, the feed may be passed through a dehydration unit to remove or reduce the water content, if any, present in the feed.

Following the transalkylation reaction, the output of the transalklyation reactor may be routed to a separation process to remove the produced benzene and polyethylbenzene from the xylene stream. The separation process can take a variety of forms, for example, boiling point distillation is a commonly employed separation technique within the industry. The boiling point of a compound is the temperature at which the vapor pressure of the liquid phase of a compound equals the external pressure acting on the surface of the liquid. Compounds generally have different, well-defined boiling points. For instance xylene has a boiling point of approximately 139° C. while benzene has a boiling point of approximately 80° C. and diethylbenzene has a boiling point of approximately 184° C. This indicates that xylene will boil at a significantly higher temperature than benzene and at a significantly lower temperature than diethylbenzene, thus providing a basis for separation of the components of the resulting stream.

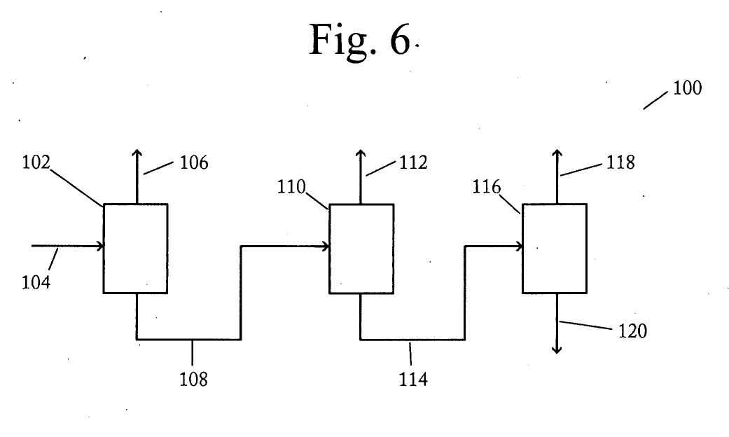

Referring to FIG. 6, in one embodiment of the separation process 100, there can be three separation zones operated under conditions known to those skilled in the art. The first separation zone 102 may include any process or combination of processes known to one skilled in the art of separation of compounds. For example, one or more distillation columns connected in series or parallel. The number of such columns may depend on the volume of the transalkylation output 104 that is the input stream to the first separation zone 102. While the operating conditions such as temperature and pressure are system specific, the first separation zone temperature may be from 80° C. to 170° C. and the first separation zone pressure may be atmospheric pressure to 50 psig, for example.

The overhead fraction 106 from this first column will generally include the lightest aromatic compounds that may be present, such as benzene or toluene, for example. Any non-aromatics that also may be present, such as for instance ethane, would also be separated with the lightest compounds. This product stream may be recovered and may be further processed in some manner, such as further separation of components. The bottom fraction 108 from this first separation zone will generally include all other heavier components that may then undergo further separation in the second separation zone 110.

The second separation zone 110 may include any process or combination of processes known to one skilled in the art of separation of aromatic compounds. For example, one or more distillation columns connected in series or parallel. The overhead fraction 112 from the second separation zone will generally include the lighter aromatic compounds such as xylene or ethylbenzene, for example. This fraction, now having reduced ethylbenzene content, may be recovered and then subsequently used for any suitable purpose such as for example, sales as a commercial grade xylene stream, or further processing, such as blending with one or more other product streams. While the operating conditions such as temperature and pressure are system specific, the second separation zone temperature may be from 100° C. to 240° C. and the second separation zone pressure may be 100 psig to 500 psig, for example.

The bottom fraction 114 from this second separation zone 110 will include the heavier aromatic compounds such as polyethylbenzenes, for example diethylbenzene. This fraction may undergo additional separation, such as in an optional third separation zone 116.

The third separation zone 116 may include any process or combination of processes known to one skilled in the art of separation of aromatic compounds. For example, one or more distillation columns connected in series or parallel. The overhead fraction 118 from the third separation zone 116 may include diethylbenzene and triethylbenzene, for example. These may be further processed, for example in a transalkylation reactor operated under conditions to convert polyethylbenzene to ethylbenzene (not shown). The bottom fraction 120 containing other heavy components may also be recovered and used for a particular purpose or subjected to further processing. While the operating conditions such as temperature and pressure are system specific, the third separation zone temperature may be from 180° C. to 240° C. and the third separation zone pressure may be atmospheric pressure to 50 psig, for example.

Various terms are used herein, to the extent a term used in not defined herein, it should be given the broadest definition persons in the pertinent art have given that term as reflected in printed publications and issued patents.

The term “alkyl” refers to a functional group or side-chain that consists solely of single-bonded carbon and hydrogen atoms, for example a methyl or ethyl group.

The term “alkylation” refers to the addition of an alkyl group to another molecule.

The term “disproportionation” refers to the removal of an alkyl group from an aromatic molecule.

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

The term “transalkylation” refers to the transfer of an alkyl group from one aromatic molecule to another.

The term “zeolite” refers to a molecular sieve containing a silicate lattice, usually in association with some aluminum, boron, gallium, iron, and/or titanium, for example. In the following discussion and throughout this disclosure, the terms molecular sieve and zeolite will be used more or less interchangeably. One skilled in the art will recognize that the teachings relating to zeolites are also applicable to the more general class of materials called molecular sieves.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Other and further embodiments, versions and examples of the invention may be devised without departing from the basic scope thereof and the scope thereof is determined by the claims that follow.

What is claimed is:

1. A method of reducing the ethylbenzene content in a stream containing xylene, the method comprising:
providing a reaction zone containing a catalyst;
introducing a feed stream comprising xylene and ethylbenzene to the reaction zone;
converting a portion of the ethylbenzene to benzene and other hydrocarbon compounds other than ethylbenzene.

2. The method of claim 1, further comprising:
removing a first product stream from the reaction zone, the first product stream having a reduced ethylbenzene content than the feed stream;
removing at least a portion of the benzene or other hydrocarbon compounds other than ethylbenzene from the first product stream to make a second product stream;
wherein the second product stream has a reduced ethylbenzene content than the feed stream.

3. The method of claim 1, wherein xylene makes up at least 25% by total weight of the feed stream.

4. The method of claim 1, wherein ethylbenzene makes up at least 25% by total weight of the feed stream.

5. The method of claim 1, wherein ethylbenzene makes up at least 40% by total weight of the feed stream.

6. The method of claim 2, wherein ethylbenzene makes up less than 25% by total weight of the second product stream.

7. The method of claim 2, wherein ethylbenzene makes up less than 18% by total weight of the second product stream.

8. The method of claim 2, wherein xylene makes up more than 75% by total weight of the second product stream.

9. The method of claim 1, wherein the catalyst has an average pore size of 6.0 angstroms or greater.

10. The method of claim 2, wherein the second product stream is within the composition specifications of a commercial grade mixed xylene product.

11. The method of claim 2, further comprising:
blending the second product stream with a third product stream containing xylene to make a fourth product stream, wherein the fourth product stream has a lower ethylbenzene content than the second product stream.

12. The method of claim 11, wherein the fourth product stream has an ethylbenzene content of less than 18% by total weight.

13. The method of claim 11, wherein the fourth product stream is within the composition specifications of a commercial grade mixed xylene product.

14. The method of claim 1, wherein the catalyst is a disproportionation catalyst.

15. The method of claim 14, wherein the disproportionation catalyst comprises a zeolite catalyst.

16. The method of claim 14, wherein the disproportionation catalyst comprises a zeolite mordenite catalyst.

17. The method of claim 14, wherein the disproportionation catalyst comprises a medal modified zeolite mordenite catalyst.

18. The method of claim 14, wherein the disproportionation catalyst comprises a zeolite nickel-mordenite catalyst.

19. The method of claim 14, wherein the reaction zone is operated at a temperature of from 65° C. to 500° C. and a pressure of between 200 psig to 1,000 psig.

20. The method of claim 1, wherein the catalyst is a transalkylation catalyst.

21. The method of claim 20, wherein the transalkylation catalyst comprises a zeolite catalyst.

22. The method of claim 20, wherein the transalkylation catalyst comprises a zeolite Y catalyst.

23. The method of claim 20, wherein the transalkylation catalyst comprises a zeolite beta catalyst.

24. The method of claim 20, wherein the reaction zone is operated at a temperature of from 180° C. to 280° C. and a pressure of between 400 psig to 800 psig.

25. A method of processing pyrolysis gasoline to produce a commercial grade xylene product, the method comprising:
providing a pyrolysis gasoline stream;
separating a first product stream comprising mixed xylene and ethylbenzene;
providing a reaction zone containing a disproportionation catalyst at disproportionation reaction conditions;
introducing the first product stream to the reaction zone;
reacting at least a portion of the ethylbenzene of the first product stream to produce benzene and other hydrocarbon compounds;
removing a second product stream from the reaction zone, the second product stream having a lower ethylbenzene content than the first product stream; and
removing at least a portion of the benzene and other hydrocarbon compounds from the second product stream to make a third product stream;
wherein the third product stream has a reduced ethylbenzene content than the first product stream.

26. The method of claim 25, wherein the third product stream has an ethylbenzene content of less than 25% by total weight.

27. The method of claim 25, further comprising:
blending the third product stream with a fourth product stream containing xylene to make a fifth product stream, wherein the fifth product stream has a lower percentage of ethylbenzene than the third product stream.

28. The method of claim 27, wherein the fifth product stream has an ethylbenzene content of less than 18% by total weight.

29. A method of converting a feed of heavy aromatics composed primarily of xylene and ethylbenzene comprising:
providing a reaction zone containing a nickel-mordenite catalyst;
introducing a first feed comprising substantially pure toluene feedstock into the reaction zone so that the first feed contacts the catalyst under initial reaction zone conditions selected for the disproportionation of substantially pure toluene to obtain a target toluene conversion between 30% and 55%;
introducing a second feed comprising xylene and ethylbenzene, allowing conversion of the second feed while the reaction zone is at the reaction zone conditions selected for the disproportionation of the pure toluene;
adjusting reactor conditions to control conversion product composition; and
removing conversion products from the reaction zone;
wherein the ethylbenzene content in the conversion products is reduced as compared to the second feed.

30. The method of claim 29, wherein the mordenite catalyst is a nickel-containing mordenite catalyst containing from 0.5% to 1.5% by weight nickel.

31. The method of claim 29, wherein the reaction zone is operated at a temperature of from 250° C. to 500° C., and a pressure of at least 200 psig.

32. The method of claim 29, further comprising:
separating the conversion products to obtain a first product stream composed primarily of xylene and ethylbenzene;
wherein the first product stream has an ethylbenzene content of less than 25% by total weight.

33. The method of claim 32, further comprising:
blending the first product stream with a second product stream containing xylene to make a third product stream, wherein the third product stream has a lower percentage of ethylbenzene than the first product stream.

34. The method of claim 33, wherein the third product stream has an ethylbenzene content of less than 18% by total weight.

35. A method of processing pyrolysis gasoline to produce a commercial grade xylene product, the method comprising:
providing a pyrolysis gasoline stream;
separating a first product stream containing mixed xylene and ethylbenzene;
providing a reaction zone containing a transalkylation catalyst at transalkylation reaction conditions;
introducing the first product stream to the reaction zone;
reacting at least a portion of the ethylbenzene of the first product stream to produce benzene and diethylbenzene;
removing a second product stream from the reaction zone, the second product stream having a reduced ethylbenzene content than the first product stream;
removing at least a portion of the benzene and diethylbenzene from the second product stream to make a third product stream;
wherein the third product stream has a reduced ethylbenzene content than the first product stream.

36. The method of claim 35, wherein the third product stream has an ethylbenzene content of less than 25% by total weight.

37. The method of claim 35, further comprising:
blending the third product stream with a fourth product stream containing xylene to make a fifth product stream, wherein the fifth product stream has a lower percentage of ethylbenzene than the third product stream.

38. The method of claim 37, wherein the fifth product stream has an ethylbenzene content of less than 18% by total weight.
